(19) United States
(12) Patent Application Publication
(10) Pub. No.: US 2019/0350491 A1
(43) Pub. Date: Nov. 21, 2019

(54) SUPPORT DEVICES FOR MEDICAL IMAGING PROCEDURES

(71) Applicant: CairnSurgical, Inc., Lebanon, NH (US)

(72) Inventors: Venkataramanan Krishnaswamy, Lebanon, NH (US); David Danielsen, Westborough, MA (US); George Bourne, Boston, MA (US)

(21) Appl. No.: 16/437,866

(22) Filed: Jun. 11, 2019

Related U.S. Application Data
(63) Continuation of application No. PCT/US2019/027315, filed on Apr. 12, 2019.
(60) Provisional application No. 62/671,597, filed on May 15, 2018.

(51) Int. Cl. A61B 5/055; A61B 5/00

(52) U.S. Cl. CPC A61B 5/0555; A61B 5/004

(57) ABSTRACT

The present disclosure includes support devices configured to be prevent breast deformation during an MRI imaging procedure. One embodiment of a support device comprises a foam insert shaped and sized to rest between a patient's breasts, generally upon the patient's sternum. The support device has a surface configured to engage an imaging modality component (i.e., coil of MRI machine) and prevent and/or minimize contact between the portion of the MRI coil and the pair of breasts when the support device is placed on the surface of the patient, which, in turn, reduces or eliminates any deformation of the breasts upon application of force from the MRI coil in a direction towards the breasts during an MRI imaging procedure. Another embodiment of a support device comprises a rigid cover including an interior cavity having sufficient volume to accommodate and surround each breast, wherein the cover is coupled to a telescoping stand resting upon the MRI table and configured to move between a variety of heights to accommodate different-sized patients. The cover is sufficiently rigid so as to shield the breasts from compression forces and thereby prevent deformation during an MRI imaging procedure.

### CROSS-REFERENCE TO RELATED APPLICATIONS

This application is a continuation of PCT/US2019/027315, filed Apr. 12, 2019, which claims the benefit of and priority to U.S. Provisional Application Ser. No. 62/671,597, filed May 15, 2018, the contents of which are incorporated by reference in their entirety.

### GOVERNMENT RIGHTS

This invention was made with Government support under NIH Grant No. R44CA210810 awarded by the National Cancer Institute under the National Institutes of Health. The Government has certain rights in this invention.

### FIELD

The present disclosure relates generally to devices for medical imaging procedures, and, more particularly, to support devices configured to be prevent breast deformation during an MRI imaging procedure.

### BACKGROUND

Magnetic resonance imaging (MRI) is a medical imaging technique used in radiology to form pictures of the anatomy and the physiological processes of the body in both health and disease. MRI scanners use strong magnetic fields, electric field gradients, and radio waves to generate images of the organs in the body. MRI is widely used in hospitals and clinics for medical diagnosis, staging of disease and follow-up without exposing the body to radiation.

Depending on the type of MRI study being performed and the specific body part undergoing imaging, different types of coils may be used. A coil helps an MRI system gather high-quality images of a specific body part. A coil generally acts an antenna which receives the MRI radio frequencies coming out of the body which is being transmitted from the MRI machine and then transmits them to a computer to be analyzed and rendered into a meaningful image. Some coils act like a frame configured to fit right over the body part being scanned, while other coils are flexible and are able to be wrapped around a body part such as the elbow, knee, or pelvis.

MRI provides one of the best imaging technologies for distinguishing soft tissue. As such, MRI of the breast offers valuable information about many breast conditions that cannot be obtained by other imaging modalities, such as mammography or ultrasound. For conventional breast MRI, a whole body coil is typically used as a transmitter coil and a smaller receiver coil is utilized to receive signals from breast tissues being examined. However, a whole body coil is not necessarily optimized or customized for breast imaging, in general, since it produces a uniform excitation around the center of the coil, this usually does not coincide with the position of breasts. Furthermore, it is generally preferable to perform MRI imaging with both an anterior and posterior coil set to enhance the quality of the images and, the quality of the image is greatly enhanced by being able to get the coils as close to the patients' anatomy as possible.

However, difficulty arises with current MRI systems in that, when a patient lies supine (i.e., lying horizontally with the face and torso facing up), the breasts fall back as a result of natural gravitational effects, and then are further compressed onto the costochondral wall (i.e. onto the rib cage) upon placement of dressings or supports between the breasts and an anterior receive coil. Such compression does not mimic how the breasts would naturally rest when the patient is lying in a supine position during most diagnostic and therapeutic procedures, as the actual location of a tissue abnormality within the breast tissue may not correspond to positioning shown in an MRI image, as the compression of the breasts may have caused the tissue abnormality to move out of its normal position. Therefore, the effectiveness of the imaging process in identifying a tissue abnormality (i.e., tumor) is compromised in that a surgeon is unable to entirely rely on an MRI image for successfully locating the tumor when performing a subsequent diagnostic (i.e., tissue biopsy) or therapeutic (i.e., ablation) procedure.

### SUMMARY

The present disclosure includes support devices configured to be prevent breast deformation by resisting compression forces applied to the breasts during an MRI imaging procedure. By preventing or reducing breast deformation, particularly during an MRI imaging procedure, any tissue abnormality within the breast tissue may generally remain in its natural resting location, particularly when the patient is lying in a supine position, rather than move as a result of the compression force. Thus, the support device of the present disclosure allows for a more accurate MRI image to be taken. More specifically, the support device will prevent a tissue abnormality from shifting from its natural resting position when the patient is lying in the supine position. Thus, the resulting MRI image will provide an accurate location of the tissue abnormality within the breast tissue, upon which a physician can rely when performing a subsequent diagnostic and/or therapeutic procedure with the patient lying in the supine position.

One embodiment of a support device consistent with the present disclosure comprises an insert shaped and sized to rest between a patient's breasts, generally upon the patient's sternum. The support device is further shaped and sized (i.e., elevated from the patient's chest) so as to provide a surface upon which a portion of a diagnostic imaging machine (e.g., a breast coil for an MRI machine) may rest, thereby reducing or completely eliminating contact between the diagnostic imaging machine and each breast and thus reducing or completely eliminating compression and deformation of each breast during the imaging procedure. The insert generally includes an upper portion configured to rest above the breasts (in cranial direction) (i.e., closer to the patient's collar bone), a lower portion configured to rest below the breasts (in caudal direction) (i.e., closer to the patient's navel area), and a central portion configured to rest between the breasts upon the patient's sternum. Each of the upper and lower portions generally flare out, such that the support device comprises an x-shape or an I-beam shape, for example. The central portion is contoured so as to generally accommodate a breast on either side to thereby maintain each breast in a relatively stationary position, even upon application of a downward/posterior compression force from dressings or supports associated with an anterior receive coil of the MRI machine. In particular, the support device is sufficiently rigid so as to provide adequate support to ensure that a breast coil remains above either breast, such that each breast is able to maintain its stationary position and resist deformation. For example, at least one of the central portion upper portion, and lower portion has a surface configured to engage a portion of the breast coil and prevent and/or minimize contact between the breast coil and each beast when the support device is placed on the surface of the patient. In turn, the support device reduces or eliminates any deformation of the breasts upon application of force from the MRI breast coil in a direction towards the breasts during an MRI imaging procedure. The support device may generally include a single size, but, depending on the size of the breasts, multiple support devices may be stacked upon one another to a desired height to accommodate larger-sized breasts so as to maintain a top surface of the support device to a sufficient height to engage a breast coil for an MRI machine and reduce or prevent contact between the breast coil and each breast. For example, smaller-sized breasts may generally require one support device (or an assembly of less support devices), as compared to larger-sized breasts, which may generally require more than one support device, including an assembly of multiple stacked support devices.

Another embodiment of a support device consistent with the present disclosure comprises a generally rigid one-piece cover including an interior cavity having sufficient volume to accommodate and surround each breast and/or chest area so as to shield the breasts from compression forces and thereby prevent deformation during an MRI imaging procedure. The cover is coupled to a telescoping stand configured to rest upon an MRI table and configured to move between a variety of heights to accommodate different-sized patients and/or different-sized breasts. The telescoping stand generally includes a base portion, which may generally be in the form of a pair of feet, for example, sufficiently spaced apart and configured to rest on either side of a patient lying in a supine position on the MRI table. The telescoping stand further includes a pair of arms coupled to the corresponding pair of feet and configured to move relative thereto between a plurality of temporarily fixed positions via an interlocking mechanism. The pair of arms are coupled to the cover. Accordingly, movement of the arms between the plurality of temporarily fixed positions with the pair of feet results in movement of the cover between a plurality of heights relative to the patient, thereby providing adjustability to accommodate a variety of different-sized patients. The interlocking mechanism between the pair of feet and pair of arms may include a quick release lever and teeth locking joints, for example. Furthermore, there may be sufficient spacing between each vertical position so as to allow the arms to slightly move so as to accommodate subtle chest rising and falling as the patient breaths. The cover may be provided in a limited number of sizes (i.e., four overall sizes) to accommodate most breast sizes.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIG. 1 is perspective view of one embodiment of a support device consistent with the present disclosure, illustrating positioning of the support device relative to a patient's breasts;

FIG. 2 is another perspective view the support device of FIG. 1 in position between the patient's breasts;

FIG. 3 is a top view of the support device of FIG. 1 in position between the patient's breasts;

FIGS. 4 and 5 are top views of another embodiment of support device consistent with the present disclosure, illustrating an exemplary spine member and a cover configured to enclose the spine member within;

FIG. 6 is a perspective view of another embodiment of a support device consistent with the present disclosure, illustrating positioning of the support device relative to a patient's breasts;

FIG. 7 is a side view of the support device of FIG. 6 positioned over the patient's breasts;

FIG. 8 is a sectional view of the support device of FIG. 6 taken along lines 8-8.

FIGS. 9A, 9B, and 9C are profile views of the cover of the support device of FIG. 6 illustrating the radii on the sides of the cover enabling the cover to fit within small bore magnetic resonance (MR) machines.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present disclosure is generally directed to support devices configured to reduce or prevent breast deformation by resisting compression forces applied to the breasts during a supine MRI imaging procedure. By preventing or reducing breast deformation, particularly during a supine MRI imaging procedure, any tissue abnormality within the breast tissue may generally remain in its natural resting location, particularly when the patient is lying in a supine position, rather than move as a result of the compression force. Thus, the support device of the present disclosure allows for a more accurate MRI image to be taken. More specifically, the support device will prevent a tissue abnormality from shifting from its natural resting position when the patient is lying in the supine position. Thus, the resulting MRI image will provide an accurate location of the tissue abnormality within the breast tissue, upon which a physician can rely when performing a subsequent diagnostic and/or therapeutic procedure with the patient lying in the supine position.

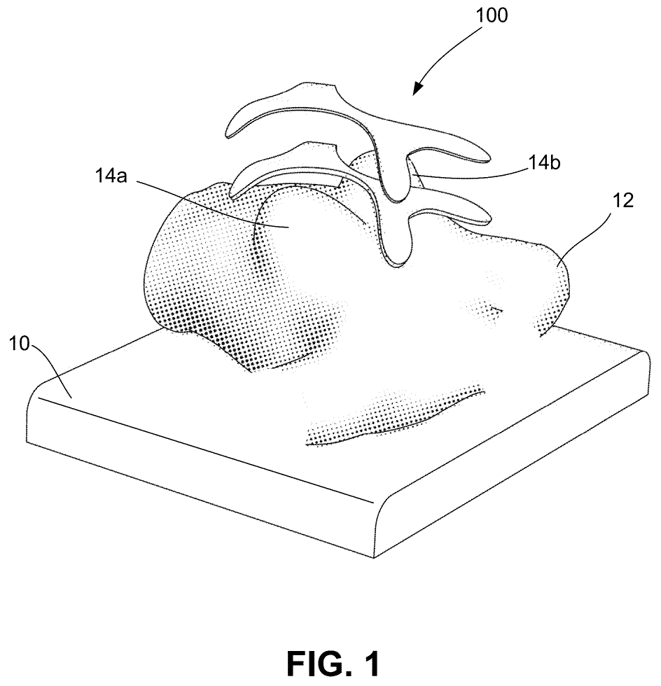

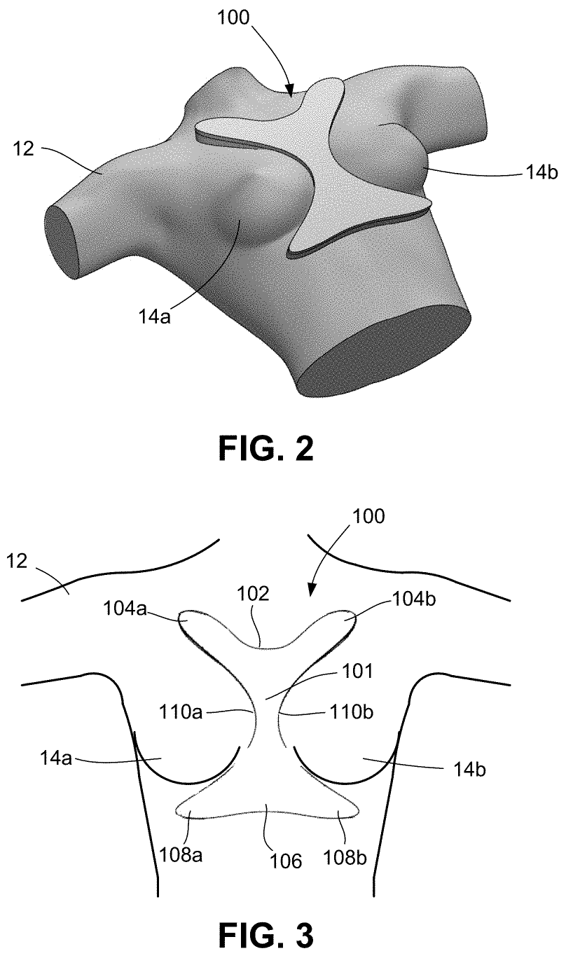

FIG. 1 is perspective view of one embodiment of a support device 100 consistent with the present disclosure. As shown, the support device 100 is configured to rest upon a patient 12 laying in a supine position upon a table 10. The support device 100 is generally shaped and/or sized to rest upon a patient's chest, generally between the breasts 14a, 14b, and provide sufficient support for a breast coil of an MRI machine to rest thereupon and further reduce and/or completely prevent contact between the breast coil and each breast so as to reduce or completely prevent the chance of compression forces being applied to the breast, which would otherwise result in deformation of the breast during an MRI imaging procedure. FIGS. 2 and 3 are perspective and top views, respectively, of the support device 100 in position between the patient's breasts 14a, 14b.

As shown, support device 100 is generally in the form of an insert having a body including a central portion 101 configured to rest between the breasts upon the patient's sternum, an upper portion 102 configured to rest above the breasts (i.e., closer to the patient's collar bone), and a lower portion 106 configured to rest below the breasts (i.e., closer to the patient's navel area). Each of the upper and lower portions 102, 106 generally flare out, such that the support device 100 comprises an x-shape or an I-beam shape. For example, the upper portion 102 includes a first pair of extensions 104a, 104b, and the lower portion 106 similarly includes a second pair of extensions 108a, 108b. The central portion 101 is contoured so as to generally accommodate a breast on either side. In particular, the central portion 101 includes opposing sides 110a, 110b, which may include an arcuate shape so as to accommodate the inner portion of each respective breast 14a, 14b. The first and second pairs of extensions 104a, 104b and 108a, 108b of the upper and lower portions 102, 106 have a corresponding arcuate shape as the opposing sides 110a, 110b of the central portion 101. In particular, the arcuate shape generally extends out from the central portion 101 to both the upper and lower portions 102, 106. Accordingly, the first pair of extensions 104a, 104b of the upper portion 102 may accommodate a top portion of each respective breast 14a, 14b, while the second pair of extensions 108a, 108b of the lower portion 106 may accommodate and effectively cradle a bottom portion of each respective breast 14a, 14b.

The support device 100 is shaped and sized so as to provide a surface upon which a portion of a diagnostic imaging machine (e.g., a breast coil for an MRI machine) may rest, thereby reducing or completely eliminating contact between the breast coil of the MRI machine and each breast, which, in turn, reduces or completely eliminates compression and deformation of each breast during the imaging procedure. For example, when the support device 100 is positioned upon the patient's chest (i.e., the central portion 101 is resting between each breast), at least one of the central portion 101, upper portion 102, and lower portion 106 may be elevated from the patient's chest to a sufficient height to thereby provide a surface upon which the breast coil of an MRI machine will rest. As such, the support device 100 is configured to receive any application of a downward/posterior compression force applied thereto from dressings or supports associated with an anterior receive coil of the MRI machine, while allowing for each breast to be maintained in a relatively stationary position during the MRI procedure.

The support device 100 may be sufficiently rigid so as to provide adequate support to resist deformation upon a compression force applied thereto, such that each breast is able to maintain its stationary position and resist deformation. The support device 100 may be composed of a sufficiently rigid polymer material, for example, which may generally be in solid foam form. However, it should be noted that the support device 100 can include any material that is sufficiently rigid and configured to provide adequate support. Furthermore, the support device 100 may be composed of a material rated for use in medical imaging procedures.

The support device 100 may generally include a single size, but, depending on the size of the breasts, multiple support devices may be stacked upon one another to a desired height to accommodate larger-sized breasts so as to maintain a top surface of the support device to a sufficient height to engage a breast coil for an MRI machine and reduce or prevent contact between the breast coil and each breast. For example, smaller-sized breasts may generally require one support device (or an assembly of less support devices), as compared to larger-sized breasts, which may generally require more than one support device, including an assembly of multiple stacked support devices. For example as shown in FIG. 1, an assembly of two inserts may be used to form the support device 100.

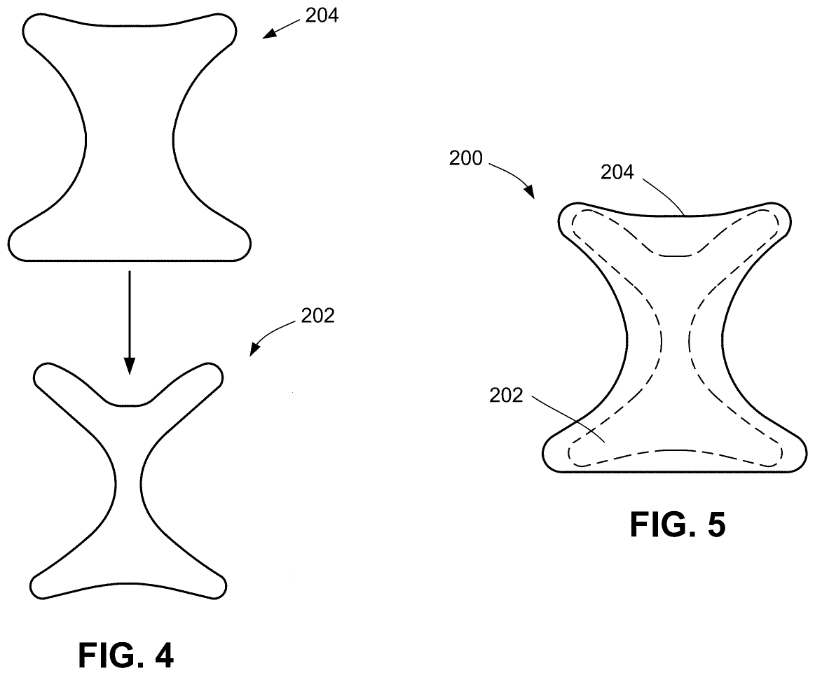

FIGS. 4 and 5 are top views of another embodiment of a support device 200 consistent with the present disclosure, which includes a spine member 202 and a cover member 204 configured to enclose the spine member 202 within. The spine member 202 generally resembles the shape of the support device 100 previously described herein, in that spine member 202 includes a central portion configured to rest between the breasts upon the patient's sternum, an upper portion configured to rest above the breasts, and a lower portion configured to rest below the breasts, and may have an x-shape or I-beam shape, generally. The cover 204 is configured to receive and enclose the entire spine member 202 within. For example, the cover 204 may include a pocket shaped and/or sized to receive the spine member 202, such that the cover 204 may simply be slid on and off of the spine member 202 when assembling/disassembling the support device 200, respectively.

The cover 204 may be disposable. For example, a new cover 204 may be used (i.e., slid onto the spine member 202) each time an imaging procedure is performed for any given patient. Once the procedure has been performed, an operator may then simply remove and discard the cover 204 and replace with a new cover 204 for the next patient. Accordingly, the support device 200 can be used for a plurality of patients over the course of a plurality of procedures, in that only the cover 204 need be replaced, while the same spine member 202 can be used from patient to patient and procedure to procedure. The spine member 202 may be sufficiently rigid so as to provide internal support for the support device 200 (i.e., resistant deformation upon a compression force applied thereto, such that each breast is able to maintain its stationary position and resist deformation). For example, the spine member 202 may be composed of a molded elastomer or polymer material. The cover 204 may be composed of a material configured to provide a cushioning effect, such as a foam material for example. Alternatively, the cover 204 may be composed of a polymer material, generally in the form of a film for sliding over the spine member 202. The cover 204 may also have an exterior surface that is shaped, sized and/or contoured in such a manner so as to generally match the surface of a patient's chest, thereby providing a form fit.

Similar to the support device 100 previously described herein, the support device 200 may generally include a single size, but, depending on the size of the breasts, multiple support devices may be stacked upon one another to a desired height to accommodate larger-sized breasts so as to maintain a top surface of the support device to a sufficient height to engage a breast coil for an MRI machine and reduce or prevent contact between the breast coil and each breast. For example, smaller-sized breasts may generally require one support device (or an assembly of less support devices), as compared to larger-sized breasts, which may generally require more than one support device, including an assembly of multiple stacked support devices.

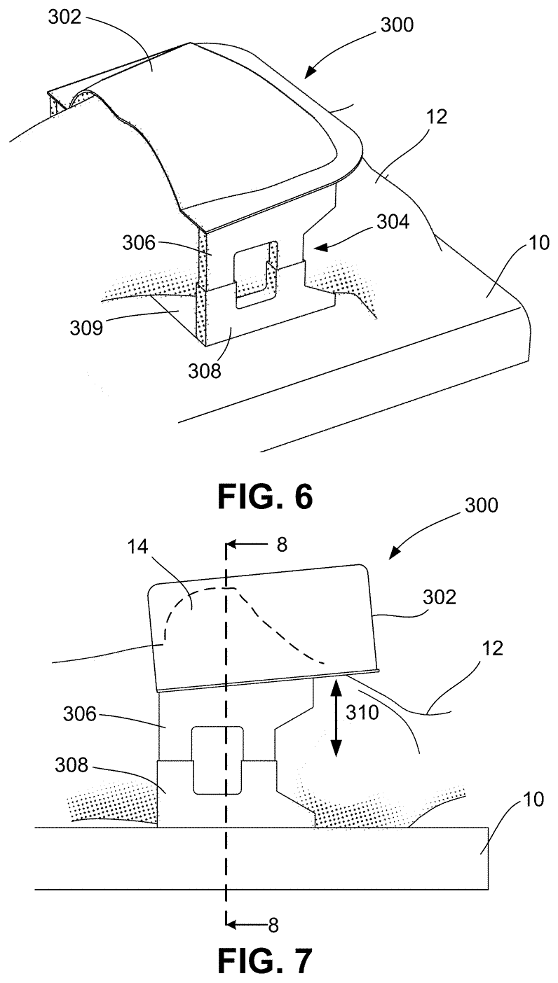

FIGS. 6 and 7 are perspective and side views, respectively, of another embodiment of a support device 300 consistent with the present disclosure. As shown, the support device 300 is configured to rest over a patient 12 laying in a supine position upon a table 10. The support device 300 includes a one-piece cover 302 including an interior cavity having sufficient volume to accommodate and surround each breast 14 so as to shield the breasts from compression forces and thereby prevent deformation during an MRI imaging procedure.

The cover 302 is coupled to a telescoping stand configured to rest upon an MRI table and configured to move between a variety of heights to accommodate different-sized patients and/or different-sized breasts. The telescoping stand generally includes a base portion 308, which may generally be in the form of a pair of feet, for example, sufficiently spaced apart and configured to rest on either side of a patient 12 lying in a supine position on the MRI table 10. The telescoping stand further includes a pair of arms 306 coupled to the corresponding pair of bases 308 with feet 309 and coupled to the cover 302.

The pair of arms 306 are configured to move relative to the corresponding pair of feet 309 between a plurality of temporarily fixed positions, as indicated by arrow 310, via an interlocking mechanism (not shown). Accordingly, movement of the arms 306 between the plurality of temporarily fixed positions with the pair of feet 309 results in movement of the cover 302 between a plurality of heights relative to the patient, thereby providing adjustability to accommodate a variety of different-sized patients. The interlocking mechanism between the pair of feet and pair of arms may include a quick release lever and teeth locking joints, for example. Furthermore, there may be sufficient spacing between each vertical position so as to allow the arms to slightly move so as to accommodate subtle chest rising and falling as the patient breathes. Additionally, or alternatively, elastic members may be included between the arms and feet the thereby allow subtle movement there between to account for patient breathing (i.e., the cover 302 to rise and fall in correspondence with rise and fall of the patient's chest).

The cover 302 may be provided in a limited number of sizes (i.e., four overall sizes) to accommodate most breast sizes. As previously described, the cover 302 may be sufficiently rigid so as to shield the breasts from compression forces and thereby prevent or reduce breast deformation during an MRI imaging procedure. The cover 302 may be composed of a sufficiently rigid polymer material, for example. However, it should be noted that the cover 302 can include any medical grade material that is sufficiently rigid and configured to provide adequate support. Furthermore, the cover 302 may be composed of a material rated for use in medical imaging procedures.

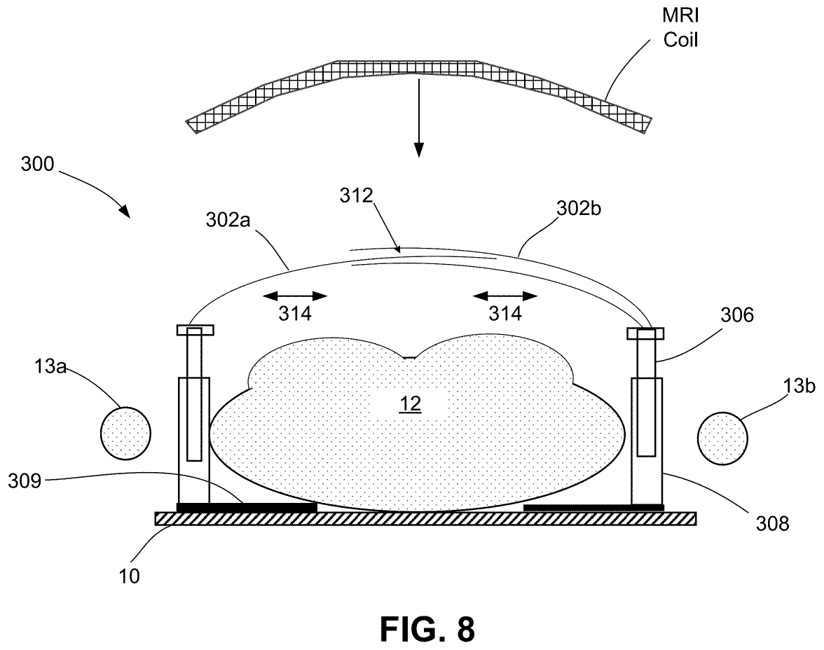

In some embodiments, the support device 300 may be configured to allow for width adjustments, in addition to height adjustments, so as to better accommodate different shapes and sizes of patients. For example,\ as shown in FIG. 8, the cover 302 may include at least a first cover portion 302a and a second cover portion 302b, wherein the cover portions 302a, 302b are configured to move in a horizontal direction while remaining coupled to one another. In one embodiment, the first cover portion 302a may be received within a cavity of the second cover portion 302b, for example, as indicated by arrow 312. As such, a user need only slide the cover portions 302a, 302b relative to one another, indicated by arrow 314, until the desired width is achieved. As further illustrated in FIG. 8, the feet 309 of the telescoping stand may be oriented in an inward direction towards the patient 12, such that the feet 309 are configured to generally rest under the patient's torso. Such a design allows for the telescoping stand 304 to generally rest between the patient's torso and arms 13a, 13b, when the patient 12 is lying on the table 10, which present a much more practical design.

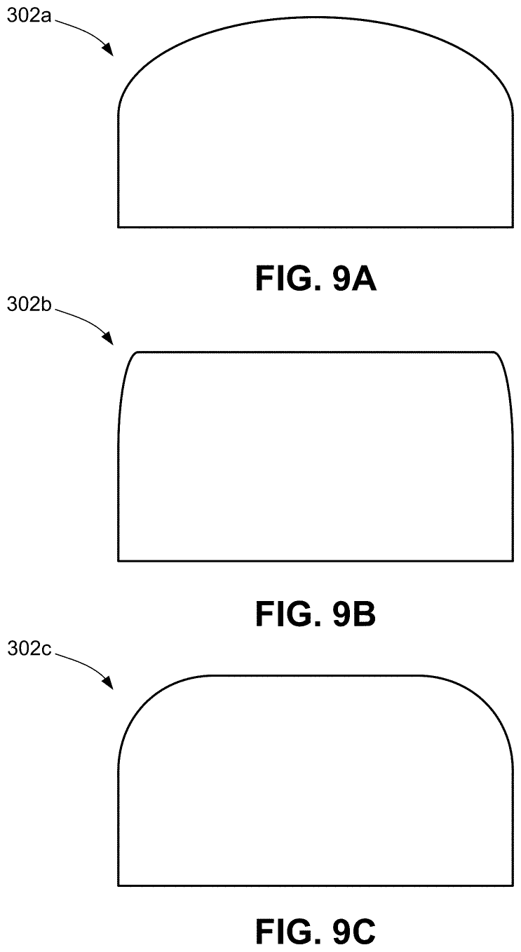

FIGS. 9A, 9B, and 9C are profile views of the cover 302 of the support device 300, illustrating different embodiments of radii on the sides of the cover 302 enabling the cover to fit within small bore magnetic resonance (MR) machines. Generally, the sides of the cover 302 may include arcuate shape to some degree, wherein the sides of the covers 302a and 302c of FIGS. 9A and 9C include a more pronounced arcuate shape, while the sides of cover 302b include a more subtle arcuate shape.

The support devices of the present disclosure are advantageous in that they are each configured to reduce or prevent breast deformation by resisting compression forces applied to the breasts during an MRI imaging procedure. By preventing or reducing breast deformation, particularly during an MRI imaging procedure, any tissue abnormality within the breast tissue may generally remain in its natural resting location, particularly when the patient is lying in a supine position, rather than move as a result of the compression force. Thus, the support devices of the present disclosure allow for a more accurate MRI image to be taken. More specifically, the support devices will prevent a tissue abnormality from shifting from its natural resting position when the patient is lying in the supine position. Thus, the resulting MRI image will provide an accurate location of the tissue abnormality within the breast tissue, upon which a physician can rely when performing a subsequent diagnostic and/or therapeutic procedure with the patient lying in the supine position.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

1. A support device for reducing and/or preventing breast tissue deformation upon compression forces applied in a direction thereto, the device comprising:
a central portion configured to rest upon a patient's sternum and comprising a pair of opposing sides shaped and/or sized to fit between a pair of breasts;
an upper portion extending from the central portion and configured to rest upon a patient's upper chest, the upper portion comprising a first pair of opposing extension members; and
a lower portion extending from the central portion and configured to rest upon a patient's abdomen, the lower portion comprising a second pair of opposing extension members;
wherein at least one of the central, upper, and lower portions has a surface configured to engage a portion of an imaging modality component and prevent and/or minimize contact between the portion of the imaging modality component and the pair of breasts when the support device is placed on the surface of the patient.

2. The device of claim 1, wherein the support device comprises a material configured to provide adequate support upon application of a compression force thereto from the imaging modality component.

3. The device of claim 2, wherein the adequate support is sufficient to maintain each breast in a relatively stationary position and resist deformation upon application of the compression thereto.

4. The device of claim 2, wherein the material comprises a sufficiently rigid polymer.

5. The device of claim 2, wherein the support device comprises a solid foam form.

6. The device of claim 1, wherein each opposing side of the central portion comprises an arcuate shape configured to accommodate an inner portion of a respective breast.

7. The device of claim 1, wherein each of the first pair of opposing extension members of the upper portion comprises an arcuate shape configured to accommodate a top portion of a respective breast.

8. The device of claim 1, wherein each of the second pair of opposing extension members of the lower portion comprises an arcuate shape configured to accommodate a bottom portion of a respective breast.

9. The device of claim 1, wherein the support device has an x-shape or I-beam shape.

10. The device of claim 1, wherein the support device is configured to be arranged in a stacked configuration with one or more other support devices to form an assembly of stacked support devices for accommodating larger breast sizes.

11-22. (canceled)
